(19) United States
(12) Patent Application Publication
(10) Pub. No.: US 2018/0149656 A1
(43) Pub. Date: May 31, 2018

(54) Regeneratable Biosensor and Methods of Use Thereof

(71) Applicant: The Charles Stark Draper Laboratory, Inc., Cambridge, MA (US)

(72) Inventors: Stephanie Angione, Somerville, MA (US); Madeline Cooper, Somerville, MA (US); Jonathan Coppeta, Windham, NH (US); Thomas Mulhern, Allston, MA (US); Hesham Azizgolshani, Belmont, MA (US)

(21) Appl. No.: 15/826,494

(22) Filed: Nov. 29, 2017

Related U.S. Application Data
(60) Provisional application No. 62/427,382, filed on Nov. 29, 2016.

(51) Int. Cl. G01N 33/574; C12Q 1/68; G01N 33/58

(52) U.S. Cl. CPC G01N 33/57488; C12Q 1/68; G01N 33/58; C12Q 2565/518; C12Q 2521/543; C12Q 2525/173; C07K 2317/32

(57) ABSTRACT

A multiplex-able, regeneratable nucleic-acid linked immunoassay method and system for the detection of a single specific, or multiple, soluble analytes in solution and regeneratable biosensor devices for same are described.

### RELATED APPLICATIONS

This application claims the benefit under 35 USC 119(e) of U.S. Provisional Application No. 62/427,382, filed on Nov. 29, 2016, which is incorporated herein by reference in its entirety.

### INCORPORATION BY REFERENCE OF MATERIAL IN ASCII TEXT FILE

This application incorporates by reference the Sequence Listing contained in the following ASCII text file:

File name: 0352-0005US1_CSDL-2549-US-01_Sequence_Listing.txt; created Nov. 29, 2017, 4 KB in size.

### FIELD OF THE INVENTION

A multiplex-able, programmable, regeneratable nucleic-acid linked immunoassay for the detection of a single specific, or multiple, soluble analytes in solution.

### BACKGROUND OF THE INVENTION

Assays for the detection and analysis of biomolecules and biomarkers such as proteins and nucleic acids in biological samples (e.g., blood, serum tissue) have been available for a number of years. Such assays include, for example, enzyme-linked imunosorbent assay (ELISA) and DNA microarrays. Some of the available methods have been adapted as biosensors for multiplex analysis with some success. However, these biosensors are designed for single-use assays on surfaces on disposable platforms or devices, which preclude the option of reusing such platforms/devices in a cost-effective manner. The ability to “re-program” or “regenerate” assay surfaces, platforms and devices for multiple use would allow the programming and re-use of the biosensor after suitable processing as an efficient cost-saving measure and also affords accurate temporal data generation, especially with automated in-line analysis.

### SUMMARY OF THE INVENTION

Described herein is a regeneratable, programmable biosensor, and methods of making and using the biosensor for detecting the presence of an analyte of interest, or a plurality of analytes of interest, in a biological sample. In particular, the biological sample is a fluid sample comprising for example, blood, serum, plasma, tissues homogenates, cellular lysates, urine, semen or cerebral spinal fluid. The analyte of interest (also referred to herein as the “target”, “target analyte” or “target molecule”) can be any analyte suitable for detection in the methods described herein and can encompass biomolecules such as proteins, peptides, and nucleic acids. One embodiment of the present invention is a regeneratable biosensor for use in analyte assays such as immunoassays.

A key aspect of the biosensor described herein is the “regeneration” and “programmability” of the biosensor. As described in detail below, the biosensor of the present invention comprises a surface or platform upon which a capture element is immobilized. The capture element is immobilized in such a manner to be stable throughout an assay process from specific capture of an analyte of interest through detection of the analyte. However, upon exposure of the biosensor to a change in conditions (e.g., exposing the biosensor to a suitable buffered wash step) a capture element can be removed and replaced with another capture element specific for assay of a different analyte of interest on the same biosensor surface, thus regenerating the biosensor for a different assay. In this sense, the biosensor is programmable, that is, the biosensor's physical properties for a specific function for a specific assay can be changed or programmed according to specific assay parameters and the biosensor reused for multiple, distinct assays.

In particular, the regeneratable biosensor of the present invention typically comprises three components: 1) a solid surface or platform such as a glass or plastic slide, microtiter plate well, a channel in a microfluidic device, or beads or microspheres, wherein the solid surface can be functionalized to immobilize one, or more, oligonucleotides onto the solid surface; 2) one, or more oligonucleotides immobilized on the functionalized solid surface that are capable of hybridizing with a complementary oligonucleotide; and 3) one, or more oligonucleotide-protein or peptide conjugates, or one, or more aptamers which are recognition/capture elements specific for the analyte of interest in the fluid sample (also referred to herein as a target molecule or biomarker).

The first component of the biosensor is a solid surface functionalized in a manner to stably immobilize an oligonucleotide. As described herein, the solid surface/platform can be of any suitable material that is capable of being functionalized, such as glass or plastic. More specifically, the solid surface must be suitable for functionalization for use as the regeneratable biosensor as described herein. In a particular embodiment, the solid surface is functionalized with (or coated with) streptavidin. Although such biotin-streptavidin attachment techniques are well known to those of skill in the art, it is important to note that some surfaces are more suitable than others for functionalization for use in a regeneratable biosensor as described herein.

The second component is one, or more, oligonucleotide(s) immobilized onto the functionalized surface. As described below, these immobilized oligonucleotides serve as “anchors” to further immobilize capture or recognition elements (comprising complementary oligonucleotides) to the biosensor surface. Such oligonucleotides can all be of the same nucleotide sequence, or of different, distinct sequences, and can be spatially arrayed/arranged on the surface in distinct patterns for multiplex assays. The oligonucleotides are typically about 20-25 nucleotides/base pairs in length, (for example, 20, 21, 22, 23, 24 or 25) but can comprise any number of nucleotides (can be of any suitable length) to form a stable, but reversible, hybridization complex with complementary oligonucleotides. For example, depending on the capture element for the target analyte to be detected, increased, or decreased, separation between the functionalized solid surface and the recognition/capture element may be desired for optimal recognition/capture/binding of the target analyte to occur, or to reduce the binding energy of the hybridization complex. Accordingly, the length of the oligo sequence can be increased or decreased to achieve the desired result. A 5′ oligo tail, such as a 5′ polyA tail, or 5′ poly G tail, of suitable length (e.g., about 7-13 nucleotides or about 8, 9, 10, 11 or 12 nucleotides) can also be attached to the oligonucleotide. In a particular embodiment of the present invention the oligonucleotides are modified for immobilization, and more specifically the oligos are biotinylated for immobilization on a streptavidin-coated surface.

The third component comprises one, or more, recognition/capture/binding elements such as a protein-oligonucleotide, or peptide-oligonucleotide conjugate wherein the protein-oligo conjugate is reversibly hybridized to the oligonucleotide(s) immobilized on the solid surface. The protein of the conjugate is a recognition element, also referred to herein as a capture or binding element, (e.g., a protein capable of capturing/binding a target analyte, an aptamer capable of binding a target analyte, or a nucleic acid such as DNA/RNA suitable for hybridizing with a target gene sequence, or nucleic acid sequence). Such a capture protein/target analyte for example, forms a binding pair, such as an antibody-antigen binding pair, a receptor-ligand binding pair, aptamer-nucleic acid binding pair or a cell-surface marker-cell binding pair). In one embodiment of the present invention, the capture element is an antibody-oligonucleotide conjugate.

In a particular, a capture antibody is covalently linked to an oligonucleotide that is of sufficient length and nucleotide complementarity to the nucleotide sequence of the oligo immobilized to the streptavidin surface resulting in the hybridization with (i.e., to) the sequence of the immobilized oligonucleotide resulting in the formation of an immobilized antibody/oligonucleotide conjugate. As described herein, the oligonucleotide is covalently linked to the protein/antibody using techniques well-known to those of skill in the art, and in particular, using “click” chemistry (see, for example, Gong et al., Bioconjugate Chem. 2016, 27, 217-225). Other attachment techniques are known to those of skill in the art.

It is important to note that, as described herein, the oligo immobilized to the functionalized surface and its complementary oligo of the recognition/capture element-oligo conjugate form a reversible hybridization complex. Using suitable reagents such as deionized water or buffer washes, under suitable conditions of temperature and time, the recognition element-oligo conjugate will de-hybridize, leaving only the oligo bound to the functionalized surface, thus regenerating the surface with the immobilized oligo(s) for subsequent use (programming) with the same, or different, recognition element-oligo conjugates, resulting in a regeneratable biosensor.

It is also important to note that while the capture/recognition element-oligonucleotide conjugate is immobilized to the solid surface, the recognition element/capture element/protein/antibody component of the conjugate remains in a structural configuration capable of binding the analyte of interest in the fluid sample, thereby forming a detectable recognition element-analyte complex bound to the biosensor surface.

A further embodiment of the present invention is a regenerative biosensor system for detecting an analyte of interest in a fluid sample, comprising the regeneratable biosensor described herein; means for contacting the biosensor with a fluid sample under conditions sufficient for the formation of a detectable antibody/analyte complex hybridized to the biotinylated oligonucleotide immobilized on the streptavidin surface of the biosensor; means for detecting the bound antibody/analyte complex; and means for washing the biosensor with suitable reagent, in a manner sufficient to de-hybridize the oligonucleotide hybridization between the antibody/analyte complex hybridized to the biotinylated oligonucleotide immobilized on the streptavidin surface of the biosensor, thereby regenerating the biosensor. In particular, the regeneratable biosensor system can be automated in a microfluidic system as described herein, and as known to those of skill in the art.

Also encompassed by the present invention are methods of detecting an analyte of interest in a fluid sample using the regeneratable biosensor described herein. The steps of the method comprise contacting a fluid sample containing the analyte of interest with the regeneratable biosensor for a time sufficient and under conditions sufficient for the analyte of interest to bind to the recognition/capture element (e.g., the protein of the protein-oligo conjugate), thereby forming an analyte-capture element-oligo conjugate (e.g., an analyte-oligo-protein conjugate) complex immobilized on the streptavidin surface. In a particular embodiment of the present invention the capture protein is an antibody, and the method is an immunoassay. The immobilized analyte-capture element-oligo conjugate is then contacted with a detectable reagent (such as a second antibody distinct from the capture antibody, also referred to herein as a detection element or detection antibody) that specifically reacts with/binds to the analyte of interest for a time sufficient and under conditions for the detectable reagent to react with the analyte. In a particular embodiment of the present invention the detectable reagent is a second antibody that binds to the analyte, for example a fluorescent-labeled antibody, or a horseradish-peroxidase labeled antibody. The final step of the method is the detection of the detectable reagent, thereby detecting the analyte of the analyte-capture element-oligo conjugate. The resulting detectable conjugate can be quantified as to approximate concentration of the analyte in the fluid sample, or qualitatively determined for the presence or absence of the analyte in the fluid sample. The fluid sample can be any fluid suitable to analysis using the biosensor of the present invention and can include, for example, blood, plasma, serum, urine, cerebral spinal fluid, and cell culture media containing cells.

The above and other features of the invention including various novel details of construction and combinations of parts, and other advantages, will now be more particularly described with reference to the accompanying drawings and pointed out in the claims. It will be understood that the particular method and device embodying the invention are shown by way of illustration and not as a limitation of the invention. The principles and features of this invention may be employed in various and numerous embodiments without departing from the scope of the invention.

### BRIEF DESCRIPTION OF THE DRAWINGS

The drawings are not necessarily to scale; emphasis has instead been placed upon illustrating the principles of the invention. The patent or application file contains at least one drawing executed in color. Copies of this patent or patent application publication with color drawing(s) will be provided by the Office upon request and payment of the necessary fee. Of the drawings:

FIG. 1 is a depiction of the regeneratable biosensor and assay method for alpha-fetoprotein.

FIG. 2 is a graph showing the results of a human alpha-fetoprotein immunoassay using the regeneratable biosensor of the present invention.

FIG. 3 is a graph showing the results of a human serum albumin immunoassay using the regeneratable biosensor of the present invention.

FIG. 4 shows a list of unique oligonucleotides and their reverse complements (SEQ ID NOS: 2-21) for use in the regeneratable biosensor of the present invention.

FIG. 5 is one depiction a microfluidic embodiment of the regeneratable biosensor (Device 1).

FIG. 6 is a second depiction the microfluidic embodiment of the regeneratable biosensor (Device 2).

FIG. 7 is a depiction of various embodiments of the microfluidic connection between the detection chambers of the microfluidic embodiments of Device 1 and Device 2.

FIG. 8 is a depiction wherein the biosensor surface comprises microsphere beads (Device 4).

FIG. 9 is a depiction of a microfluidic embodiment (Device 5) wherein the biosensor surface comprises magnetic beads.

FIG. 10 is a depiction of another embodiment of the biosensor wherein the surface comprises optical fibers (Device 6).

### DETAILED DESCRIPTION OF THE PREFERRED EMBODIMENTS

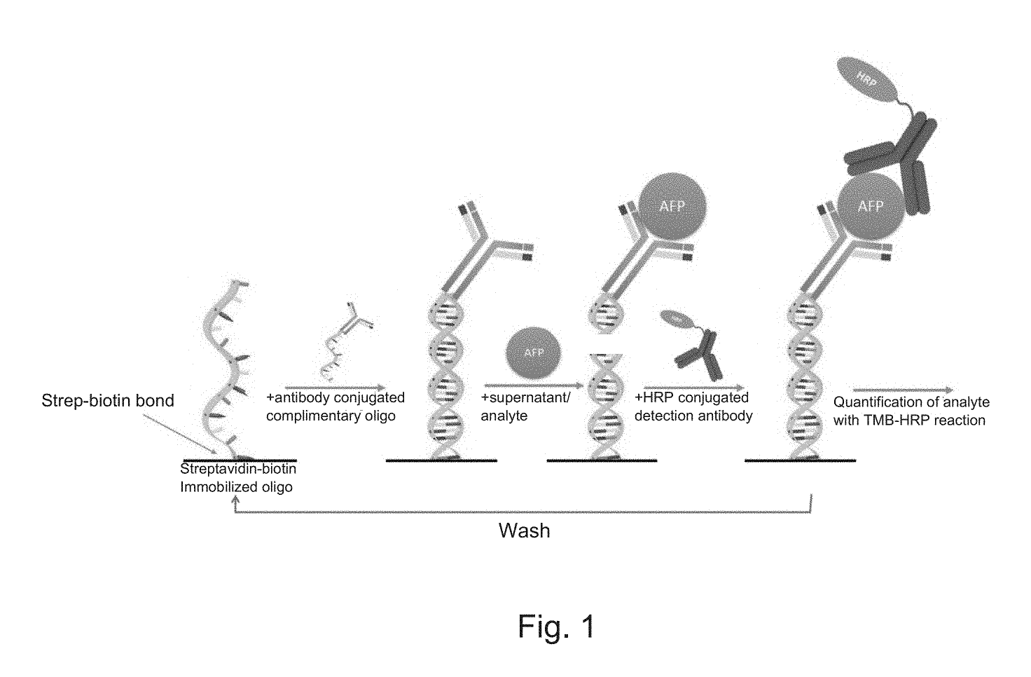

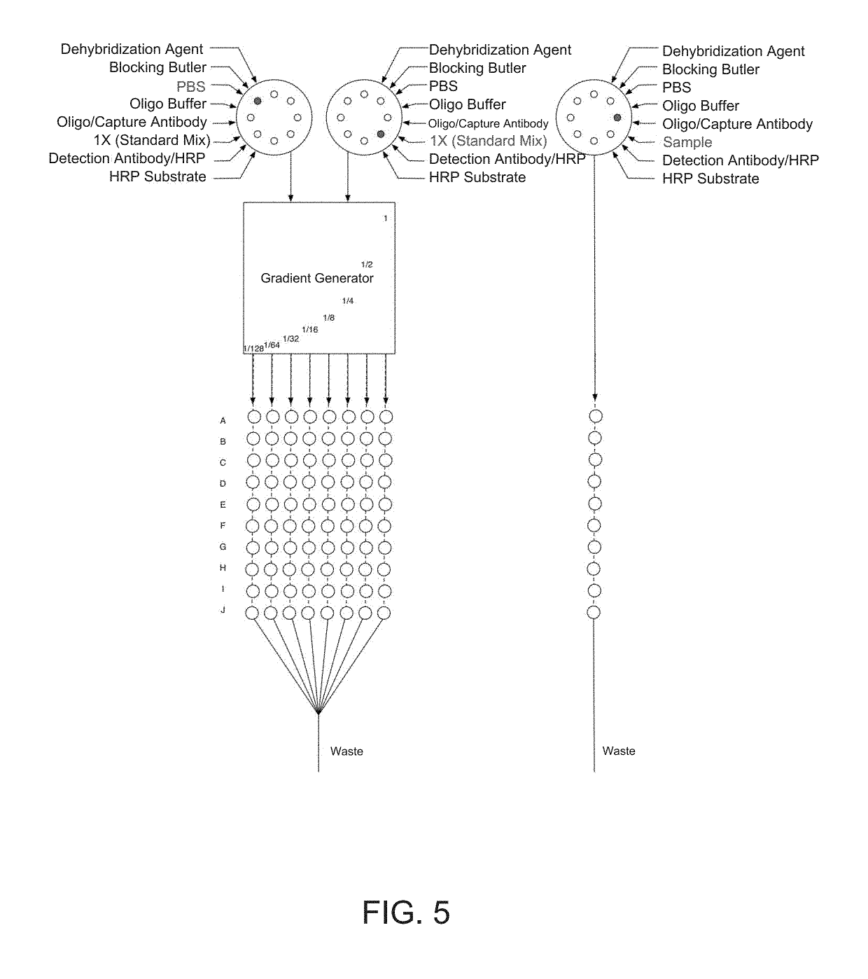

The present invention describes a regeneratable biosensor suitable for use in immunoassay methods with results comparable to single-use sandwich ELISAs. As described herein, the term “RELISA” encompasses such ELISA immunoassays using the regeneratable biosensor of the present invention. In particular, the present invention utilizes short unique oligo sequences that are covalently linked to specific antibodies, which can then be hybridized, and immobilized using the reverse complement of the oligo sequence bound to a surface (FIG. 1). For example, a biotinylated reverse complement oligo can be immobilized on a streptavidin coated surface. An antibody-coupled oligo can be then be reversibly bound to the surface by the oligo-reverse complement oligo hybridization. Incubation with the specific antigen of interest, followed by incubation with a labeled detection antibody provides the readout of analyte detection. Making use of the fact that dsDNA cannot hybridize properly in the absence of essential stabilizing cations, both monovalent and divalent, the immobilized antibody-oligo conjugates can be washed away (de-hybridized) with a simple rinse with deionized H₂O. This allows the same surface to be reused repeatedly with either the same antibody-oligo conjugate, or a different antibody conjugated to the same oligo. Spatial patterning of unique reverse complement oligo sequences on a microfluidic chip enables a multiplexed measurement of a range of different analytes. A microfluidic device can be designed (see for example, the devices depicted in FIGS. 5-10) to automate the wash and measurement steps on a spatially patterned microfluidic chip or other surface such as a bead or fiber. Repeated measurements allow for temporally resolved measurements of biological phenomena. The benign chemistry may allow in-line sensors in biological systems.

### Generation of Oligo-Antibody Conjugates

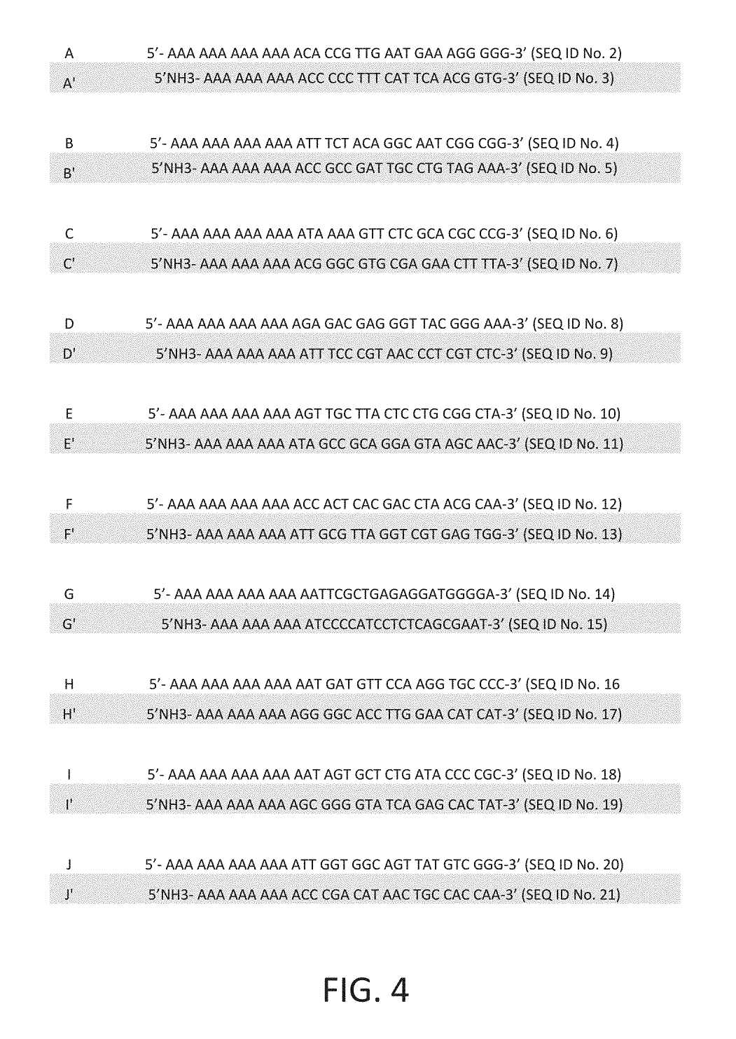

A short (e.g., 20-25 base pair) oligo sequence can be designed for use in the biosensor described herein. The oligos can comprise poly nucleotide tails, for example, a polyA or polyG tail. As one example, an oligo with a 5′ polyA sequence of 10 nucleotides (AAA AAA AAA ATA CGG ACT TAG CTC CAG GAT (SEQ ID NO:1) and a 5′ azide group was covalently coupled to antibodies using click chemistry detailed in Gong et al., 2015. Other oligos suitable for use in the present invention are shown in FIG. 4 as SEQ ID NOS: 2-21. Additional oligos can be designed by one of skill in the art.

Briefly, strain promoted alkyne-azide cycloaddition (SPAAC) chemistry was performed by activating azide-free antibody with 4 molar excess of DBCO-PEG5-NHS. NHS reacts with an amine group on the antibody, DBCO provides the alkyne group for the subsequent cycloaddition to the oligo, and PEG5 serves to reduce steric hindrance and increases solubility of the DBCO compound for improved conjugation efficiency. Next, this DBCO-antibody is reacted with 4 molar excess of the azide containing oligo to perform the alkyne-azide cycloaddition. The conjugated oligo is the reverse complement of the oligo immobilized on the RELISA surface. Unbound DBCO and oligo are removed from reaction using Amicon Ultra-0.5 Centrifugal Filter units with NMWL of 50-100 kDa.

Other conjugation chemistries are known to those of skill in the art and can include, for example, commercially available kits such as Thunder-Link from Innova Biosciences.

### Immobilization of Oligo-Antibody Conjugates on a Surface

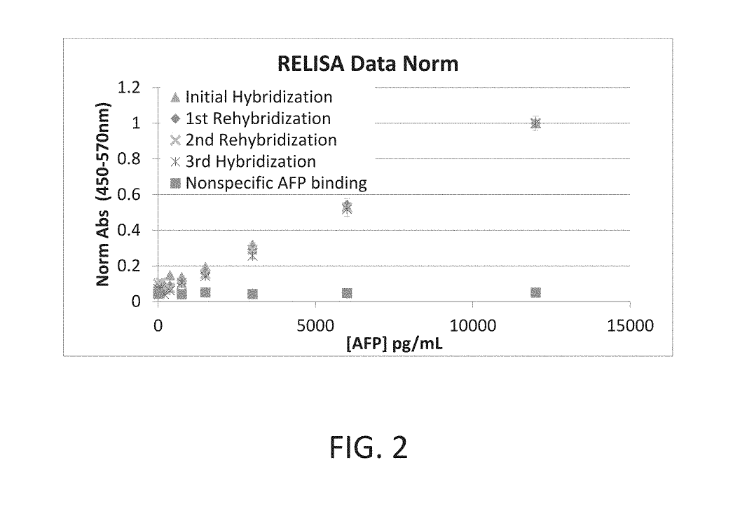

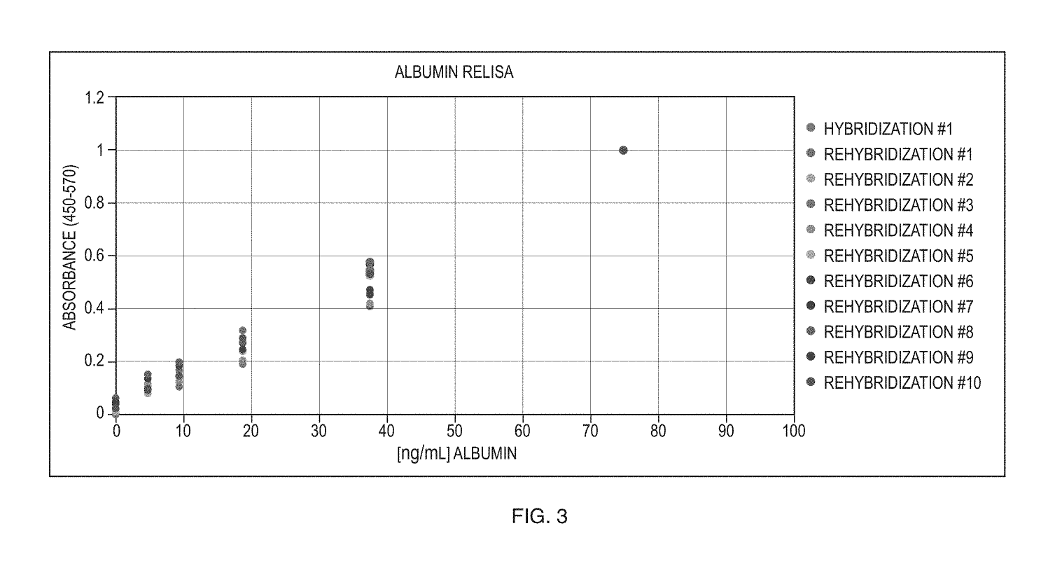

The regeneratable biosensor of the present invention comprises a solid surface, or platform, and can include, for example, plates, wells, microfluidic device channels, beads and optical fibers. In particular, any surface suitable for functionalization with a reactive moiety can be used in the biosensor. In particular, the surface is suitable for strepavidin coating as described herein. As shown in Table 1, numerous surfaces were tested for suitability of use for the regeneratable biosensor. Other surfaces can be evaluated for suitable use using the techniques described herein. RELISA results shown in FIGS. 2 and 3 were generated using a commercially-available coated microplate and the strepavidin-biotin coupling modality, however as shown in Table 1, other surfaces/substrate and coupling modalities can also be used.

TABLE 1
Survey of Surface Chemistries

| | | Chemical Coupling | |
|---|---|---|---|
| Treatment | Substrate | Modality | Comments |
| Peirce High Capacity | Polystyrene | Streptavidin - Biotin | Successful hybridization |
| Streptavidin Coated | | | Successful dehybridization |
| Microplates | | | |
| Promega SAM² ® Biotin | Commercial | Streptavidin - Biotin | Successful hybridization |
| Capture Membranes | membrane | | No dehybridization |
| High capacity | Polystyrene | Streptavidin - Biotin | Successful hybridization |
| polymerized | | | No dehybridization |
| streptavidin | | | |
| BioTez Poly-strep R kit | Polystyrene | Streptavidin - Biotin | Successful hybridization |
| | | | No dehybridization |
| Arrayit streptavidin | Glass | Streptavidin - Biotin | Successful hybridization |
| | | | No dehybridization |
| MicroSurfaces, Inc | Glass | Streptavidin - Biotin | No dehybridization |
| Streptavidin coated | | | |
| slides | | | |
| Silanized Glass (APTES + | Glass | Silane | No dehybridization |
| Glutaraldehyde + | | | |
| Animated Oligo) | | | |
| Carboiimide coupling | Polystyrene | EDC-NHS | No dehybridization |
| Biomat/Immunosurfaces | Polystyrene, | Streptavidin - Biotin | No dehybridization |
| | Cyclic Olefin | | |
| | Copolymer | | |
| Schott | Glass | Streptavidin - Biotin | No dehybridization |
| Artic White | Polystyrene | Streptavidin - Biotin | Successful hybridization |
| | | | Successful dehybridization |
| Custom Streptavidin | Polystyrene | Streptavidin - Biotin | Successful hybridization |
| Coating | | | Successful dehybridization |

For example, the reverse complementary oligo sequence with a 5′ biotin moiety followed by a polyA nucleotide sequence was immobilized using streptavidin on a well plate surface in hybridization buffer. See for example, the oligonucleotides listed in FIG. 4). The hybridization buffer consists of 150 mM NaCl, 0.25% Tween-20 and 0.1% bovine serum albumin (BSA), and optionally 5-10 mM MgCl2, at pH 7.5. Other suitable buffers can be determined by one of skill in the art. The oligo-antibody conjugate can then be hybridized to the immobilized reverse complement in the same hybridization buffer via incubation at a time and temperature suitable for the hybridization reaction to occur (e.g., room temperature for 1 hour). This step effectively immobilizes the antibody on the plate surface and the unbound fraction can be washed away with hybridization buffer where the number of washes are sufficient to remove the unbound antibodies, (e.g., two-three times) A round of successful DNA hybridization can be ensured by hybridizing a fluorescent reverse complement oligo in parallel wells and detecting fluorescence on a standard plate reader.

### Aptamers

Also encompassed by the present invention is a variation of the biosensor described herein that includes using an aptamer as the recognition element. For example, instead of use of a capture protein, the recognition element/capture element can be a specific oligo sequence that can detect an analyte of interest. This would not require covalent coupling of the immobilization oligo sequence to the recognition element (i.e. antibody) but would simply consist of the two specific sequences in a continuous DNA polynucleotide. The detection element (i.e. secondary antibody) can also be an aptamer, labeled with a fluorophore or other moiety.

### RELISA Procedure

Following immobilization, the analyte of interest (e.g., an antigen) in the sample can be incubated with (or contacted with) the immobilized conjugates under conditions sufficient to ensure specific interaction such as binding of antigen to antibody, binding of ligand to receptor protein or hybridization. The binding complex of analyte/immobilized conjugate is then washed in hybridization buffer and incubated with a detection moiety labeled secondary antibody. Following detection, the oligo-antibody conjugate and resulting bound analytes can be removed from the surface with a suitable wash buffer, such as in RNase/DNase free water. This leaves only the streptavidin bound biotinylated oligo on the surface of the biosensor substrate, allowing for re-use of the biosensor for detection of other analytes with different oligo-antibody conjugates. De-hybridization can also be achieved in alkaline conditions by washing with a basic solution, for example, IM NaOH, or by generating pH changes with electrolysis. Ease of de-hybridization can also be tuned based on the length of the complementary oligos.

### RELISA Data

Following the above procedure using an antibody to human alpha-fetoprotein (AFP), it is demonstrated that the surface can be regenerated at least four times for a successful and reproducible sandwich ELISA against AFP (FIG. 2). Furthermore, when higher concentrations of AFP are used to continue the standard curve toward saturation, the characteristic ELISA “S” curve is seen, indicating that the regeneratable platform is comparable to classic single-use sandwich ELISAs.

Following the above procedure using an antibody to human serum albumin (ALB), it is demonstrated that the surface can be regenerated at least 10 times for a successful and reproducible sandwich ELISA against ALB (FIG. 3). Furthermore, when higher concentrations of ALB are used to continue the standard curve toward saturation, the characteristic ELISA “S” curve is seen, indicating that the regeneratable platform is comparable to classic single-use sandwich ELISAs.

### Device

### Materials/Surface Functionalization:

The biosensor surface/chip can comprise an optically clear glass or hard plastic surface, like polystyrene or COC, or a polystyrene bead, magnetic bead, microsphere or fiber such as an optical fiber. A surface suitable for the biosensor of the present invention allows surface functionalization with a suitable reactive moiety using well-established methods in the field. The surface will also facilitate quantitative and/or quantative optical readouts. An example, of an optical readout can be wave length absorbance or flourescence. In particular, functionalization of the surface can be coating the surface with a reactive moiety at a surface concentration or density suitable for use in the biosensor described herein. More specifically, for example, functionalization can be coating/absorbing streptavidin on the surface using techniques known to those of skill in the art. For use in the biosensor of the present invention, streptavidin density on the surface of e.g., microplates can be in the range of about 1-1.5×10⁻¹² mol/mm², and, more particularly, the density is about 1.25×10⁻¹² mol/mm². Evaluation of the density of streptavidin coating can be determined by known techniques. Examples of some combinations of substrates/treatments and coupling modalities are described in Table 1.

With streptavidin as the reactive moiety coating the surface, a biotinylated oligo can then be immobilized on the surface through the strong biotin-streptavidin interaction. The biotinylated oligos are immobilized or contacted with (e.g., printed on) the biosensor surface in a spatially suitable pattern in a detection region or area of the biosensor. The pattern of biotinylated oligos immobilized on the biosensor surface/substrate can be any pattern where a unique oligo is immobilized and separated from each other oligo at a specific and sufficient distance to allow hybridization of capture elements (e.g., antibodies) to each immobilized oligo. The biotinylated oligos can be patterned/organized on a detection region of the biosensor using a variety of methods including microprinting or microfluidic channels to flow unique oligos in parallel patterns. Alternatively, as described below, the biotinylated oligos can be patterned on microbeads. Streptavidin functionalized can be completed on hard plastic if the surface is appropriately treated prior to adsorption (see for example, Table 1).

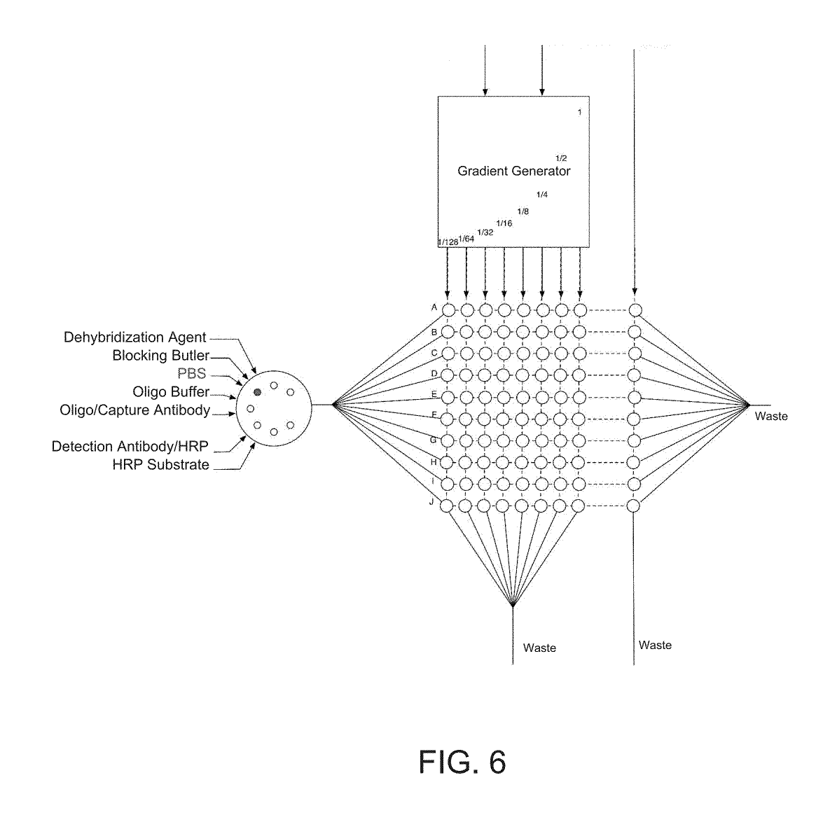

One embodiment of the disclosed invention is the use of RELISA in a microfluidic device that can automate the detection of a panel of analytes of interest. The use of microfluidic embodiments of the invention can reduce required sample volumes containing the analytes of interest by approximately an order of magnitude. FIG. 5 (Device 1, or Dev 1) and 6 (Device 2, or Dev 2) depict two embodiments of the microfluidic sensor. As depicted in FIG. 5 Dev 1, and FIG. 6 Dev 2, the microfluidic RELISA sensor can comprise a standard calibration region and a sample detection region. In both embodiments of Dev 1 and Dev 2, the standard calibration region comprises a gradient generator that upon receiving a standard mixture of analytes of interest (e.g., in a fluid sample) allows for generation of streams of distinct concentrations of a desired profile akin to those used to create the standard curves for a typical ELISA. The gradient generator can take embodiments similar to those used, for example, by Campbell and Groisman, Lab Chip, 2007:7, 264-272. In the embodiments of Dev 1 and Dev 2, the arrays of circles depict detection regions which are connected by microfluidic channels.

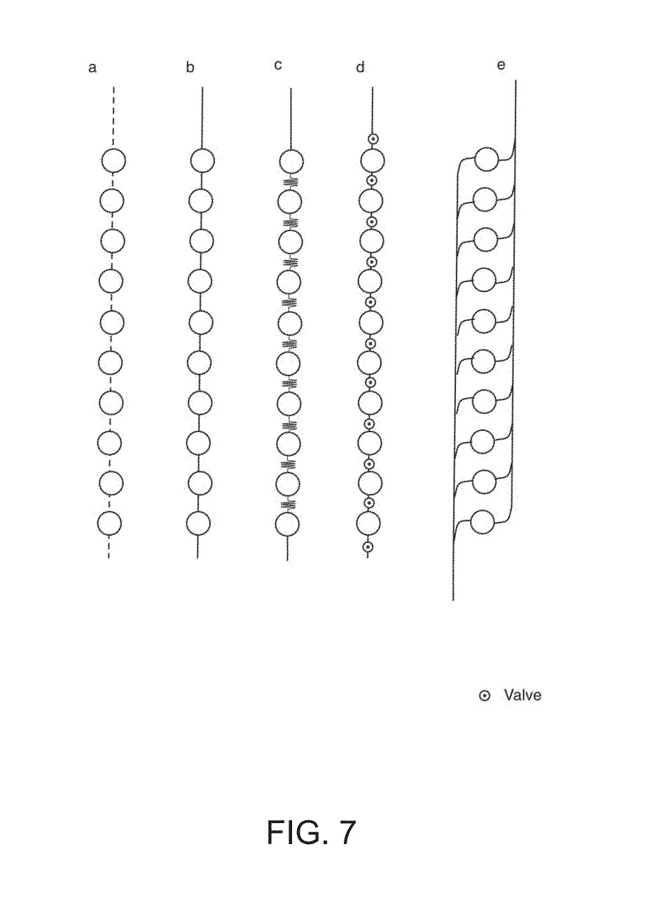

The microfluidic channels connecting the detection regions, here depicted as dashed lines, can take various embodiments as depicted in FIG. 7, Device 3 (Dev 3). These configurations include but are not limited to straight channels, serpentine or tortuous channels, channels separated with valves, and interlaced channels. It should also be noted that the detection regions are not limited to the arrangements and geometrical form factors depicted herein, and can further take alternate embodiments. In the embodiments of Dev 1 and Dev 2, each row of oligos in the detection region—here labeled A-J—represents a unique oligomer to allow hybridization of different antibodies onto the surface of each region, hence allowing detection of a panel of different analytes of interest. The distinct oligomers A-J are chosen such that they only hybridize with their matched conjugate and show minimal cross reactivity to the unmatched conjugates. FIG. 4 lists unique oligomer sequences that can be used to program the embodiments described here. It should be noted that the number of unique analytes to be detected is not limited to 10 as described in these embodiments.

In the devices of the present invention, the delivery of the reagents can be integrated using chip valves and pumps, and the microfluidic sensor can have reservoirs of the various reagents in a variety of forms including prefilled cartridges. For example, in the embodiment of Device 1, the fluid handling of the calibration region and that of the sample are separate while in the embodiment of Device 2, the two regions share the same fluid handling. In the embodiment of Device 2, the row-wise microfluidic connection between the detection regions allows for delivery of the distinct oligomers to each row for the purpose of programming the microfluidic sensor. The microfluidic embodiments described here can utilize a variety of signal amplification methods including but not limited to HRP based or circular DNA amplification techniques. Furthermore, depending on the generated signal, a variety of detection mechanisms can be utilized. These detection mechanisms can include a variety of optical methods such as optical density measurements, luminescence, and fluorescence measurements in both transmitted and reflected modes, as well as electrochemical methods when using electroactive substrates.

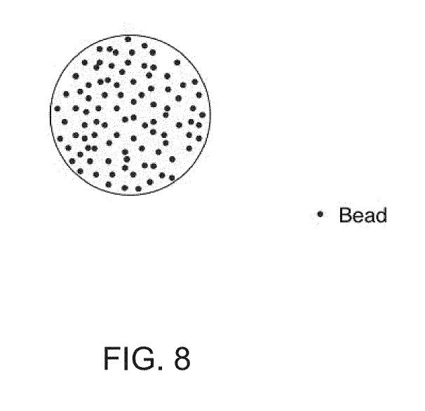

In another embodiment of the present invention, a device is depicted in FIG. 8, (Device 4, or Dev 4). In Device 4, the oligo sequences are attached are on the surface of microsphere beads, including but not limited to polystyrene beads that are addressed to individual RELISA reaction chambers of embodiments such as those described in Dev. 1 and Dev. 2, according to the oligomer sequence coating the beads.

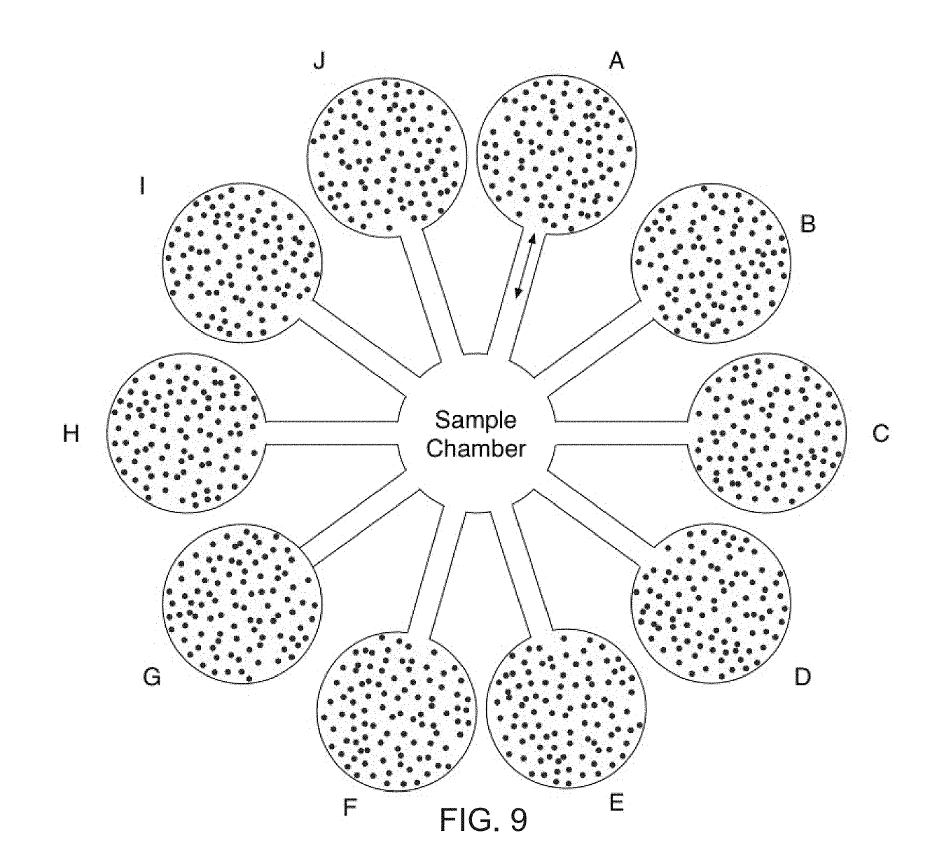

In FIG. 9, another embodiment (Device 5 or Dev 5) is depicted, where the biosensor comprises a sample chamber connected to a number of reservoir chambers containing magnetic beads coated with unique oligomers. In this embodiment, the beads from each chamber are moved sequentially to the sample chamber with the aid of electromagnetic force, where, if present, the analyte of interest binds the capture antibodies on the coated beads. These beads are subsequently moved back to their respective reservoir chamber, where the signal is amplified and detected as described previously.

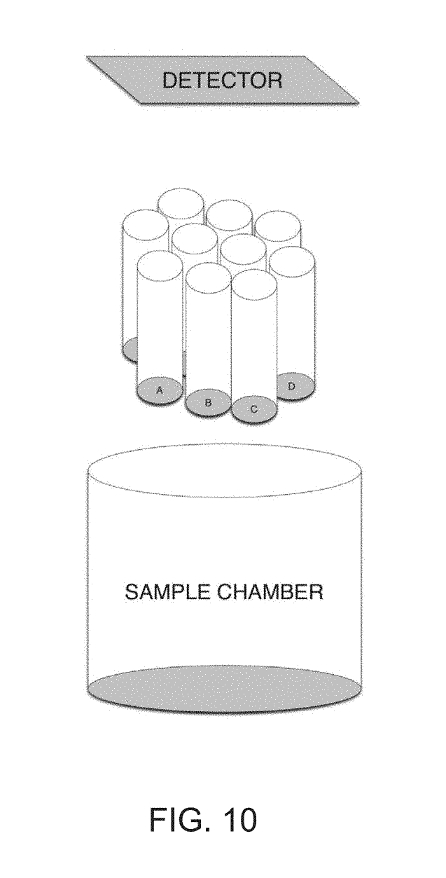

In yet another embodiment, depicted in FIG. 10, Device 6 (or Dev 6), the RELISA sensor consists of an array of optical fibers, the ends of which constitute the RELISA surface, and are in optical connection with an appropriate detector. Each fiber is coated with a unique oligomer sequence with the appropriate conjugated antibody or aptamer, which subsequently binds the analyte of interest if present. The array of fibers is moved to a different chamber where detection of analytes of interest occurs. Alternatively, this fiber optic embodiment could be used for measurements in multi-well plates where sample volume is small (e.g. 96 or 384). The use of an optical fiber facilitates the measurement of a small volume without significant loss of sample volume. For example, a 384 array of fiber bundles could be dipped into a 384 well plate and then brought to a device that is similar to previously described devices (for example, Dev 1 or 2) to go through the wash and detection steps.

Applications

The biosensors and RELISA methods of detection as described herein can be used to isolate and identify a wide variety of analytes of interest, including proteins, exosomes, and cell free DNA in different matrices. These matrices include cell culture medium, urine, and blood. The analytes can detect biomarkers for disease, and can be expanded as a panel of markers, so as to identify inflammation, cancer progression, diarrheal disease, rhinovirus/influenza virus, and bacterial infections. Additionally, the panel arrays can be used to detect markers of tissue differentiation. For example, in detecting differentiation markers, the progression of tissue development from iPSC cells to mature human tissue can be monitored by detecting biomarkers of immature cells such as alpha fetoprotein or CYP3A7 substrates or metabolites, or biomarkers of mature cells such as albumin, alpha 1 anti-trypsin and CYP3A4 substrates and metabolites

While this invention has been particularly shown and described with references to preferred embodiments thereof, it will be understood by those skilled in the art that various changes in form and details may be made therein without departing from the scope of the invention encompassed by the appended claims.

What is claimed is:

1. A regeneratable biosensor for detecting the presence of an analyte, or a plurality of analytes, of interest in a fluid sample, comprising:
a) a functionalized solid surface, wherein the functionalized surface is capable of immobilizing an oligonucleotide;
b) one, or more, oligonucleotides, wherein the oligonucleotide is immobilized on the functionalized surface;
c) one, or more, capture elements covalently linked to an oligonucleotide, wherein the oligo nucleotide sequence is complementary to the sequence of the oligonucleotide immobilized on the functionalized surface and the oligonucleotide linked to the capture element is reversibly hybridized to the immobilized oligonucleotide, thereby forming an immobilized capture element-oligonucleotide conjugate, and wherein the capture element-oligonucleotide conjugate is capable of capturing the analyte of interest in the fluid sample, thereby forming a detectable capture element-oligo-analyte complex bound to the biosensor surface.

2. The biosensor of claim 1 wherein the capture element is selected from the group consisting of: a protein, a peptide, an antibody, an aptamer or a nucleic acid sequence.

3. The biosensor of claim 1 wherein the oligonucleotides immobilized on the functionalized surface are spatially arranged on the surface.

4. A regeneratable biosensor for detecting the presence of an analyte, or a plurality of analytes, of interest in a fluid sample, comprising:
a) a solid surface coated with streptavidin;
b) one, or more, biotinylated oligonucleotides, wherein the biotinylated oligonucleotide is immobilized on the streptavidin surface;
c) one, or more, antibodies covalently linked to an oligonucleotide, wherein the oligo nucleotide sequence is complementary to the sequence of the oligonucleotide immobilized on the streptavidin surface and the oligonucleotide linked to the antibody is reversibly hybridized to the immobilized oligonucleotide, thereby forming an immobilized antibody/oligonucleotide conjugate, and wherein the antibody/oligonucleotide conjugate is capable of binding the analyte of interest in the fluid sample, thereby forming a detectable antibody/analyte complex bound to the biosensor surface.

5. A regenerative biosensor system for detecting an analyte, or a plurality of analytes, of interest in a fluid sample, comprising:
a) the biosensor of claim 1;
b) means for contacting the biosensor with the fluid sample under conditions sufficient for the formation of a detectable capture element-analyte complex hybridized to the oligonucleotide immobilized on the functionalized surface of the biosensor;
c) means for detecting the bound capture element-analyte complex; and
d) means for washing the biosensor with suitable reagent and in a manner sufficient to de-hybridize the oligonucleotide hybridization between the capture element-analyte complex hybridized to the oligonucleotide immobilized on the surface of the biosensor, thereby regenerating the biosensor.

6. A regenerative biosensor system for detecting an analyte, or a plurality of analytes, of interest in a fluid sample, comprising:
a) the biosensor of claim 4;
b) means for contacting the biosensor with the fluid sample under conditions sufficient for the formation of a detectable antibody-analyte complex hybridized to the oligonucleotide immobilized on the streptavidin surface of the biosensor;
c) means for detecting the bound antibody-analyte complex; and
d) means for washing the biosensor with suitable reagent and in a manner sufficient to de-hybridize the oligonucleotide hybridization between the antibody-analyte complex hybridized to the biotinylated oligonucleotide immobilized on the streptavidin surface of the biosensor, thereby regenerating the biosensor.

7. A method of detecting an analyte, or a plurality of analytes, of interest in a fluid sample, the method comprising:
a) contacting a fluid sample containing the analyte of interest with the regeneratable biosensor of claim 1 for a time sufficient and under conditions sufficient for the analyte of interest to bind to the capture element of the capture element-oligo conjugate, thereby forming an analyte-capture element-oligo conjugate complex immobilized on the solid surface;
b) contacting the analyte-capture element-oligo conjugate of step a) with a detectable reagent that specifically reacts with/binds to the analyte of interest for a time sufficient and under conditions for the detectable reagent to react with the analyte; an
c) detecting the reagent, thereby detecting the analyte of the analyte-capture element-oligo conjugate.

8. The method of claim 7 wherein the fluid sample is selected from the group consisting of: blood, plasma, serum, urine, cerebral spinal fluid, cells, cell culture media containing cells, exosomes, microvesicles and circulating nucleic acids.

9. The method of claim 7 wherein the detectable reagent is a detectably-labeled antibody that binds to the analyte.

10. A method of detecting an analyte, or a plurality of analytes, of interest in a fluid sample, the method comprising:
a) contacting a fluid sample containing the analyte of interest with the regeneratable biosensor of claim 4 for a time sufficient and under conditions sufficient for the analyte of interest to bind to the antibody of the antibody-oligo conjugate, thereby forming an analyte-antibody-oligo conjugate complex immobilized on the streptavidin surface;
b) contacting the analyte-antibody-oligo conjugate of step a) with a detectable reagent that specifically reacts with/binds to the analyte of interest for a time sufficient and under conditions for the detectable reagent to react with the analyte; and
c) detecting the reagent, thereby detecting the analyte of the analyte-antibody-oligo conjugate.

11. The method of claim 10 wherein the fluid sample is selected from the group consisting of: blood, plasma, serum, urine, cerebral spinal fluid, cells, cell culture media containing cells, exosomes, microvesicles and circulating nucleic acids.

12. The method of claim 10 wherein the detectable reagent is a detectably-labeled antibody that binds to the analyte.


SEQUENCE LISTING (21 sequences)

SEQ ID NO: 1 - DNA, 30 bp, Unknown
  Synthetic
aaaaaaaaaatacggacttagctccaggat

SEQ ID NO: 2 - DNA, 33 bp, Unknown
  Synthetic
aaaaaaaaaaaaacaccgttgaatgaaaggggg

SEQ ID NO: 3 - DNA, 30 bp, Unknown
  Synthetic
aaaaaaaaaaccccctttcattcaacggtg

SEQ ID NO: 4 - DNA, 33 bp, Unknown
  Synthetic
aaaaaaaaaaaaatttctacaggcaatcggcgg

SEQ ID NO: 5 - DNA, 30 bp, Unknown
  Synthetic
aaaaaaaaaaccgccgattgcctgtagaaa

SEQ ID NO: 6 - DNA, 33 bp, Unknown
  Synthetic
aaaaaaaaaaaaataaaagttctcgcacgcccg

SEQ ID NO: 7 - DNA, 30 bp, Unknown
  Synthetic
aaaaaaaaaacgggcgtgcgagaactttta

SEQ ID NO: 8 - DNA, 33 bp, Unknown
  Synthetic
aaaaaaaaaaaaagagacgagggttacgggaaa

SEQ ID NO: 9 - DNA, 30 bp, Unknown
  Synthetic
aaaaaaaaaatttcccgtaaccctcgtctc

SEQ ID NO: 10 - DNA, 33 bp, Unknown
  Synthetic
aaaaaaaaaaaaagttgcttactcctgcggcta

SEQ ID NO: 11 - DNA, 30 bp, Unknown
  Synthetic
aaaaaaaaaatagccgcaggagtaagcaac

SEQ ID NO: 12 - DNA, 33 bp, Unknown
  Synthetic
aaaaaaaaaaaaaccactcacgacctaacgcaa

SEQ ID NO: 13 - DNA, 30 bp, Unknown
  Synthetic
aaaaaaaaaattgcgttaggtcgtgagtgg

SEQ ID NO: 14 - DNA, 33 bp, Unknown
  Synthetic
aaaaaaaaaaaaaattcgctgagaggatgggga

SEQ ID NO: 15 - DNA, 30 bp, Unknown
  Synthetic
aaaaaaaaaatccccatcctctcagcgaat

SEQ ID NO: 16 - DNA, 33 bp, Unknown
  Synthetic
aaaaaaaaaaaaaatgatgttccaaggtgcccc

SEQ ID NO: 17 - DNA, 30 bp, Unknown
  Synthetic
aaaaaaaaaaggggcaccttggaacatcat

SEQ ID NO: 18 - DNA, 33 bp, Unknown
  Synthetic
aaaaaaaaaaaaaatagtgctctgataccccgc

SEQ ID NO: 19 - DNA, 30 bp, Unknown
  Synthetic
aaaaaaaaaagcggggtatcagagcactat

SEQ ID NO: 20 - DNA, 33 bp, Unknown
  Synthetic
aaaaaaaaaaaaattggtggcagttatgtcggg

SEQ ID NO: 21 - DNA, 30 bp, Unknown
  Synthetic
aaaaaaaaaacccgacataactgccaccaa